(19) United States
(12) Patent Application Publication
(10) Pub. No.: US 2025/0283988 A1
(43) Pub. Date: Sep. 11, 2025

(54) ULTRASOUND DIAGNOSTIC APPARATUS

(71) Applicant: FUJIFILM Corporation, Tokyo (JP)

(72) Inventor: Kenichi ADACHI, Tokyo (JP)

(73) Assignee: FUJIFILM Corporation, Tokyo (JP)

(21) Appl. No.: 19/059,050

(22) Filed: Feb. 20, 2025

(30) Foreign Application Priority Data
Mar. 5, 2024 (JP) 2024-032637

(51) Int. Cl. G01S 7/52; A61B 8/00; G01S 15/89

(52) U.S. Cl. CPC G01S 7/5209; G01S 7/52022; G01S 15/8915; A61B 8/4494

(57) ABSTRACT

In related-art processing, phasing processing for generating a large number of reception parallel beams was performed in a period of a length equal to a period in which data of a reception signal for a large number of track pulses is written in a channel (CH) memory. Meanwhile, in processing of the embodiment, a period of the phasing processing was lengthened by using not only the writing period to the CH memory but also an idle period for charging required for push pulse transmission and an idle period for cooling a probe. As a result, the number of reception beams that can be generated in the period of the phasing processing can be increased as compared with the related art.

### CROSS REFERENCE TO RELATED APPLICATION

This application claims priority to Japanese Patent Application No. 2024-032637 filed on Mar. 5, 2024 which is incorporated herein by reference in its entirety including the specification, claims, drawings, and abstract.

### BACKGROUND OF THE DISCLOSURE

### 1. Field of the Disclosure

The present disclosure relates to an ultrasound diagnostic apparatus, and particularly relates to reception signal processing of an ultrasonic wave.

### 2. Description of the Related Art

As the reception signal processing of the ultrasonic wave, reception parallel beam processing, pulse inversion (phase inversion), and the like are known. In the reception parallel beam processing, a plurality of reception beams are formed in parallel from one transmission (that is, one transmission beam). In the pulse inversion, ultrasonic waves are transmitted using two transmission signals having phases inverted with each other, and a reception signal obtained from one transmission signal and a reception signal obtained from the other transmission signal are subjected to addition processing or difference processing.

JP2017-077311A discloses a circuit configuration suitable for the reception signal processing involving synthesis processing such as pulse inversion and the reception parallel beam processing. In this circuit configuration, a reception signal set for one beam processed by each reception processing unit is stored in each reception signal memory. Each synthesis processing unit reads out a reception signal set corresponding to each beam number and performs synthesis processing to generate a synthetic reception signal corresponding to the beam number. A synthetic reception signal of each vibration element is stored in each synthetic signal memory. Each synthetic signal memory comprises two storage regions corresponding to two beams. A phasing addition unit reads out a plurality of synthetic reception signals corresponding to a plurality of vibration elements from a storage region corresponding to the beam number in accordance with a delay pattern corresponding to each beam number. The phasing addition unit generates a reception beam signal by performing delay processing and addition processing on the plurality of synthetic reception signals.

In addition, there is a technique called ultrasound elastography that uses ultrasonic waves to measure and image a physical quantity related to elasticity of a biological tissue. In shear wave elastography, which is a type of the ultrasound elastography, a tissue is excited to generate a shear wave, nd the propagation of the shear wave is tracked by an array of ultrasound transducers to obtain a propagation velocity of the shear wave. The tissue is excited by, for example, a high-intensity ultrasonic pulse from an array of transducers. This ultrasonic pulse for excitation is called a push pulse. That is, the push pulse is a pulse for generating a shear wave.

In addition, the ultrasonic pulse used for tracking the shear wave is called a track pulse. The track pulse can be said to be a pulse for observing a shear wave. In order to capture the propagation of the shear wave with a high resolution, the track pulse is repeatedly transmitted and received a large number of times (for example, several hundred times) for one shear wave.

For example, JP2012-010099A discloses an ultrasound diagnostic apparatus comprising an elasticity value calculation unit that measures a propagation velocity of a transverse wave generated in a measurement region of a biological tissue of a subject by a push pulse emitted from an ultrasound probe to the measurement region to obtain an elasticity value of the measurement region, an elastic image generation unit that generates an elastic image based on the elasticity value obtained by the elasticity value calculation unit, and a display unit that displays the elastic image generated by the elastic image generation unit.

### SUMMARY OF THE DISCLOSURE

Since the reception parallel beam processing has a certain degree of processing load, in a case in which the number of reception beam signals to be generated for one transmission is to be increased, it is necessary to either introduce high-performance hardware or increase a processing time.

The present disclosure provides a technique for increasing the number of reception beam signals generated by phasing addition in an ultrasound diagnostic apparatus that excites a tissue with a transducer array to generate a shear wave and that tracks the shear wave with the transducer array.

An ultrasound diagnostic apparatus disclosed in the present specification comprises: a transducer array having a plurality of transducers; a transmission and reception circuit that causes the plurality of transducers to perform transmission and reception according to a transmission and reception sequence including a first transmission and reception period in which an ultrasonic wave for generating a shear wave is transmitted and received, a second transmission and reception period in which an ultrasonic wave for observing a shear wave is transmitted and received a plurality of times, and an idle period in which no ultrasonic wave is transmitted; a first storage unit that is provided for each transducer and stores, for each transmission of the ultrasonic waves for observing a shear wave in the second transmission and reception period, a reception signal through the transducer in response to the transmission; a second storage unit that is provided for each transducer and has at least two line memories used to store the reception signals corresponding to different transmissions of the ultrasonic waves for observing a shear wave, the reception signals being read out from the first storage unit; and a phasing addition unit that generates a plurality of reception beam signals by performing phasing addition for each of a plurality of different delay patterns on a set of the reception signals for the plurality of transducers corresponding to the same transmission, the set of the reception signals being read out from the line memories of the second storage unit provided for each transducer, according to the delay pattern, in which phasing addition unit executes a process of generating the plurality of reception beam signals corresponding to one transmission, by using at least a part of a time having a length corresponding to the idle period in addition to a time having a length corresponding to the second transmission and reception period.

According to this configuration, a phasing addition period can be made longer than in a configuration in which a plurality of reception beam signals are generated in a phasing addition period having a length equal to the second transmission and reception period.

Here, the idle period may include a charging period required for transmitting the ultrasonic wave for generating a shear wave from end of the second transmission and reception period until start of the first transmission and reception period in the next transmission and reception sequence.

In addition, the idle period may include a cooling period for cooling the transducer array after the second transmission and reception period.

In addition, the ultrasound diagnostic apparatus may further comprise: a generation unit that generates, before each of the individual transmissions in the second transmission and reception period starts, a parameter for the phasing addition for the reception signal corresponding to the transmission, and the second storage unit may be configured in an internal memory in the same integrated circuit as the phasing addition unit, whereas the first storage unit may be configured in an external memory externally attached to the integrated circuit, and the parameter generated by the generation unit may be temporarily stored in the external memory, and is read from the external memory to the internal memory in a case of being used for the phasing addition.

In this configuration, a large amount of control parameters for a large number of reception beam signals to be generated by transmission and reception of the ultrasonic waves for observing a shear wave are held in the external memory having a much larger capacity than the internal memory provided in the integrated circuit in general.

According to the present disclosure, it is possible to increase the period for the phasing addition as compared with the related art, and thus it is possible to increase the number of reception beam signals that can be generated in that period.

### BRIEF DESCRIPTION OF THE DRAWINGS

FIG. 1 is a diagram showing an example of a functional configuration of an ultrasound diagnostic apparatus.

FIG. 2 is a diagram showing an example of a time chart of transmission and reception processing of an ultrasonic wave in a shear wave elastography mode.

FIG. 3 is a diagram for describing a transition of a display mode of the ultrasound diagnostic apparatus.

FIG. 4 is a diagram for describing a process of generating a shear wave elastography image.

### DESCRIPTION OF THE EMBODIMENTS

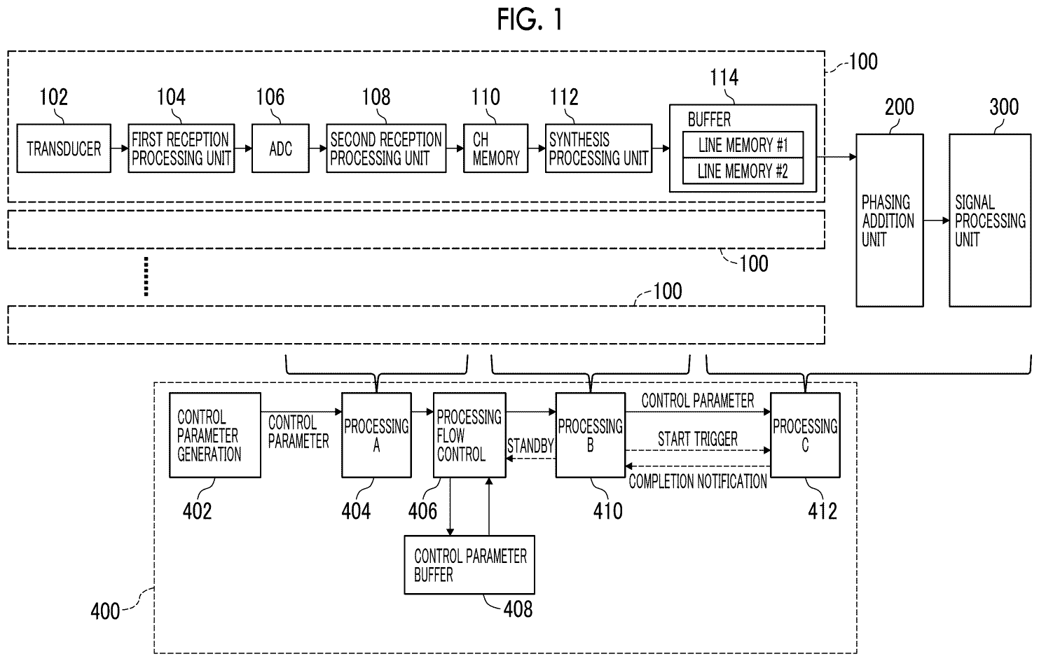

FIG. 1 is a diagram showing an example of a functional configuration of an ultrasound diagnostic apparatus in the present disclosure. A transducer 102 is an element that transmits and receives ultrasonic waves. A transducer array in which a plurality of the transducers 102 are arranged is formed, and the transducer array is provided in an ultrasound probe. The transmission and reception of the ultrasonic waves by means of the transducer array are controlled to form an ultrasonic beam, electronic scanning with the ultrasonic beam is performed. Examples of the electronic scanning method include electronic linear scanning and electronic sector scanning. Incidentally, the ultrasound probe is used by being placed in contact with a surface of a living body or being inserted into a body cavity of the living body.

The plurality of transducers 102 constituting the transducer array are controlled in transmission by a transmission unit (not shown) that functions as a transmission beam former. Then, a reception signal obtained by each transducer 102 receiving the ultrasonic wave from the living body is subjected to signal processing in each unit in a subsequent stage shown in FIG. 1. Several functional elements that process the reception signal for each transducer 102 are provided in the subsequent stage of the plurality of transducers 102. A group of functional elements for each transducer 102 is called a channel 100. One channel 100 includes the transducer 102, a first reception processing unit 104, an A/D converter (ADC) 106, a second reception processing unit 108, a channel (CH) memory 110, a synthesis processing unit 112, and a buffer 114. The ultrasound diagnostic apparatus has a channel 100 for each transducer 102 provided in the ultrasound probe.

The first reception processing unit 104 adjusts (for example, amplifies) a gain of a reception signal output from a corresponding transducer 102. The reception signal of which the gain is adjusted by the first reception processing unit 104 is input to a corresponding ADC 106. The ADC 106 converts an analog reception signal into a digital reception signal.

The second reception processing unit 108 executes necessary reception processing on the digital reception signal. Specific examples of the reception processing include decimation (thinning-out processing). The number of samples of the digital reception signal is thinned out to, for example, n/m (n and m are natural numbers) by decimation. The reception signal (digital) processed by the second reception processing unit 108 is stored in the CH memory 110.

The CH memory 110 stores the reception signal obtained from the corresponding transducer 102 and processed by the second reception processing unit 108. The CH memory 110 stores a reception signal set for one beam (that is, a set of reception signals corresponding to one beam number) related to the transducer 102. A specific example of the reception signal set is a set of a reception signal obtained by one transmission signal and a reception signal obtained by the other transmission signal in pulse inversion.

The CH memory 110 is a relatively large capacity memory capable of storing a reception signal set for one beam, and can be implemented using, for example, a dynamic random access memory (DRAM). The CH memories 110 of a plurality of the channels 100 may be mounted on the same one storage device (for example, one package of DRAM) or may be mounted by combining a plurality of storage devices (for example, a plurality of packages of DRAM).

The synthesis processing unit 112 reads out the reception signal set stored in the corresponding CH memory 110 and performs synthesis processing. The CH memory 110 stores a reception signal set for one beam corresponding to one beam number among a plurality of beam numbers. The synthesis processing unit 112 reads out the reception signal set corresponding to each beam number stored in the corresponding CH memory 110 and performs synthesis processing to generate a synthetic reception signal corresponding to the beam number.

For example, in a case in which a specific example of the reception signal set is a set of two reception signals obtained by pulse inversion, the synthesis processing unit 112 performs addition processing on the two reception signals. Through this addition processing, for example, a synthetic reception signal of a secondary harmonic (even harmonic) is formed. A synthetic reception signal in which, for example, the even harmonic is reduced (or removed) may be formed from a difference between the two reception signals obtained by the pulse inversion.

The buffer 114 comprises two line memories #1 and #2 corresponding to two beams (two beam numbers). Then, of the synthetic reception signals for two beams, a synthetic reception signal corresponding to one beam number can be written into one line memory #1 or #2, while a synthetic reception signal corresponding to the other beam number can be read out from the other line memory #2 or #1. That is, the buffer 114 has a function as a ping-pong buffer.

The buffer 114 may be configured by using, for example, a dual-port memory configured of a static random access memory (SRAM). For example, the buffers 114 of the plurality of channels 100 may be implemented by one device (for example, one package of storage device) or may be implemented by combining a plurality of devices (for example, a plurality of packages of storage devices).

In addition, main signal processing elements (for example, the second reception processing unit 108 and the synthesis processing unit 112 of each channel 100 described above, and a phasing addition unit 200 and a signal processing unit 300 described below) of the ultrasound diagnostic apparatus may be integrated on one integrated circuit. As such an integrated circuit, for example, a field programmable gate array (FPGA) or an application specific integrated circuit (ASIC) can be used. The buffer 114 may be implemented using an internal memory (for example, an SRAM) on the integrated circuit. On the other hand, considering a case in which the reception signals corresponding to a large number of beam numbers are stored, the CH memory 110 may be mounted on an external memory having a large capacity that is externally attached to the integrated circuit. In addition, not only the signal processing elements but also a processor that performs control and other information processing may be further integrated on the integrated circuit.

An apparatus configuration in which the synthesis processing unit 112 is not provided is also possible. For example, in an apparatus configuration in which pulse inversion is not performed, only one reception signal corresponding to one transmission signal is stored in the CH memory 110, and the synthesis processing unit 112 that synthesizes a plurality of reception signals is not necessary. In this case, the reception signal that is temporarily stored in the CH memory 110 is directly transferred to the buffer 114.

The phasing addition unit 200 generates a reception beam signal by performing delay processing and addition processing on a plurality of synthetic reception signals corresponding to the plurality of transducers 102. The phasing addition unit 200 reads out the synthetic reception signal of each channel generated for each beam number and stored in each buffer 114, and generates a reception beam signal (that is, reception beam data) corresponding to each beam number.

According to a delay pattern corresponding to each beam number, the phasing addition unit 200 reads out the synthetic reception signal from the line memory #1 or #2 corresponding to the beam number. For example, data of an address corresponding to the delay pattern is read out from the synthetic reception signal for one beam stored in a corresponding line memory #1 or #2. The delay processing (that is, phasing processing) is implemented by the readout address control. Then, a reception beam signal is formed by adding data that has been subjected to phasing addition according to the delay pattern from the plurality of synthetic reception signals.

In addition, the phasing addition unit 200 has a function of executing reception parallel beam processing of forming a plurality of reception beam signals for each beam number. The reception parallel beam processing of the ultrasound diagnostic apparatus of FIG. 1 may be the same processing as the processing in the related art (for example, disclosed in JP2017-077311A). For example, M (M is a natural number) phasing addition units 200 may be provided and each phasing addition unit 200 may execute the reception parallel beam processing, thereby increasing the number of reception parallel beams by M times.

In this way, phases of the reception signals of the plurality of transducers 102 are aligned with respect to a focus point, and electronic focusing and electronic beam steering are achieved.

The reception beam signal generated by the phasing addition unit 200 is further processed in the signal processing unit 300 in the subsequent stage. The signal processing unit 300 executes various types of signal processing required to generate an image of each mode of the ultrasound diagnostic apparatus. For example, in a B-mode, processing such as detection and logarithmic compression is performed on the reception beam signal. In a color flow mapping mode (color Doppler mode), for example, processing such as autocorrelation calculation is executed on a complex signal. In a case in which the Doppler mode is selected, processing required for extracting Doppler information and performing frequency analysis, such as quadrature detection processing is executed. In addition, in a case where shear wave elastography display is instructed, transmission and reception control and signal processing for generating the shear wave elastography image, such as calculation of a propagation velocity of a shear wave from a reception signal of a tracking pulse, are executed. Hereinafter, the shear wave elastography will be abbreviated as SWE. SWE is an abbreviation for Shear Wave Elastography.

The detection processing (including quadrature detection processing) may be executed for each transducer 102 before the phasing addition processing of the phasing addition unit 200. In addition, by converting the reception signal into a baseband signal through the detection processing, the number of samplings for digitization can be generally reduced. Therefore, for example, a thinning-out rate in decimation may be further increased (i.e. the number of data items to be thinned out may be increased as compared with a case in which detection is not performed).

Then, for example, image data of an ultrasound image is formed through interpolation processing, coordinate conversion processing, or the like by a digital scan converter, and the ultrasound image corresponding to the image data is displayed on a display device such as a liquid crystal monitor.

The element group related to the signal processing of the reception signal has been described above.

A controller 400 controls the circuit configuration for the signal processing on the reception signal described above and implements the generation of various diagnostic images described above. The controller 400 comprises each functional unit of control parameter generation 402, processing A 404, processing flow control 406, a control parameter buffer 408, processing B 410, and processing C 412. The controller 400 may be implemented using, for example, a processor or a memory that is integrated on the same integrated circuit as the signal processing element group described above.

The control parameter generation 402 generates a control parameter required for processing such as reception beam generation in each digital signal processing element downstream of the ADC 106 in each channel 100. The control parameter generation 402 receives information such as apparatus operation information and transmission and reception mode information from a main controller of the ultrasound diagnostic apparatus, and generates a control parameter based on the information.

The control parameter generated by the control parameter generation 402 includes, for example, the thinning-out number, a storage destination of the reception signal, a storage destination of a focus calculation parameter, the number of parallel beams, and beam attribute information required for image generation in various modes in the signal processing unit 300.

The thinning-out number is referred to in the thinning-out processing of the second reception processing unit 108.

The storage destination of the reception signal is a memory address of the CH memory 110 in which the reception signal processed by the second reception processing unit 108 is stored. For example, in a case in which the CH memory 110 is secured in the external memory, a memory address allocated to the CH memory 110 in a memory space managed by an operating system of the controller 400 is one control parameter.

Similarly, the storage destination of the focus calculation parameter is a memory address of a storage destination of a parameter generated to calculate the focus of the reception beam. The storage destination is also secured in, for example, the external memory.

The number of parallel beams is the number of parallel beams generated for one transmission in the reception parallel beam processing.

The beam attribute information required for image generation in various modes in the signal processing unit 300 includes, for example, a beam type (for example, a black and white B-mode or a tracking pulse), a direction of a reception beam in the B-mode, and an order of reception beams in the SWE mode.

The control parameter generation 402 generates, for example, for each transmission of the ultrasonic wave, a group of control parameters applied to the reception signal generated by the transducer 102 of each channel 100 for the transmission. A timing at which the control parameter generation 402 generates a group of control parameters to be applied for certain transmission may be any time before the transmission, and does not have to be immediately before the transmission. For example, in a case of executing m-th ultrasonic transmission (m is an integer of 1 or more), a group of control parameters for (m+n)-th ultrasonic transmission (n is a predetermined integer of 2 or more) may be generated. In addition, the control parameter generation 402 may collectively generate the control parameters for a plurality of times of transmission. In addition, the control parameter generation 402 may of course generate a control parameter applied in common to a plurality of times of transmission in addition to the control parameter each transmission.

The processing A 404 is processing of processing (for example, thinning-out processing) the reception signal in digital format output from the ADC 106 by means of the second reception processing unit 108 and writing digital data (for example, reception signal data after thinning-out), which is a result of the processing, to the CH memory 110. Here, the second reception processing unit 108 executes signal processing, such as the thinning-out processing, by using, for example, one or more specific control parameters (for example, a value of the thinning-out number) corresponding to the second reception processing unit 108 among the group of control parameters generated by the control parameter generation 402.

The processing flow control 406 reads and writes the control parameter to and from the control parameter buffer 408. That is, the processing flow control 406 writes the control parameter generated by the control parameter generation 402 in the control parameter buffer 408. In addition, the processing flow control 406 reads out necessary control parameters from the control parameter buffer 408 for the synthesis processing unit 112, the phasing addition unit 200, the signal processing unit 300, and the like at a timing necessary for the processing of the respective units, and provides the control parameters to the respective units.

In one example, in the processing A 404, the second reception processing unit 108 may directly read out the control parameter generated by the control parameter generation 402 on an internal memory on an integrated circuit such as an FPGA, from the internal memory, and use the control parameter. In this case, the second reception processing unit 108 does not need to read out the control parameter from the control parameter buffer 408.

The control parameter buffer 408 is a buffer that stores the control parameter. In the present embodiment, in order to generate an SWE image, it is necessary to store the reception signal data and the control parameters corresponding to a very large number of times of the track pulse transmission (for example, several hundred times) in the CH memory 110 and the control parameter buffer 408. This is to respond to a case in which the ultrasound diagnostic apparatus displays the SWE image in a superimposed manner on, for example, a B-mode tomographic image. That is, in this case, one of the line memories #1 and #2 of the buffer 114, which is the ping-pong buffer, is occupied for a long time by the reception signal data in the B-mode. This is because the B-mode requires a long reception time and generates a large number of parallel beams. Therefore, until the line memory #1 or #2, which stores the reception signal in the B-mode, is freed, the next line (that is, the reception signal) cannot be read, and thus the phasing addition processing for the SWE cannot proceed. Therefore, for example, during a period in which the phasing addition is being executed to create the B-mode tomographic image, the reception signals obtained by each track pulse for the SWE need to be accumulated in the CH memory 110. In addition, for the same reason, it is necessary to hold the control parameter corresponding to the reception signal for the SWE during the period in which the phasing addition is being executed to create the B-mode tomographic image. As described above, in order to generate the SWE image, it is necessary to track the propagation of the shear wave using a very large number of track pulses, and the amount of data of the control parameters corresponding to the track pulses is also quite large. Therefore, in a model in which the capacity of the internal memory on the integrated circuit on which the signal processing elements and the like are integrated is small, a large number of control parameter groups for the SWE are not be accommodated in the internal memory in some cases. In order to respond to such a case, for example, the control parameter buffer 408 may be secured on an external memory externally attached to the integrated circuit, similarly to the CH memory 110.

The processing B 410 is processing of performing synthesis processing on the reception signal data read out from the CH memory 110 by means of the synthesis processing unit 112 and writing data resulting from the processing to the line memory #1 or #2 in the buffer 114. Here, the synthesis processing unit 112 is in a standby state in a case in which both the line memories #1 and #2 in the buffer 114 are full. In a case in which the next data can be written in one of the line memories #1 and #2, the synthesis processing unit 112 acquires control parameters for the next reception signal data from the control parameter buffer 408 via the processing flow control 406, and executes the synthesis processing using the control parameters. In the example shown in the drawing, the group of control parameters read out from the control parameter buffer 408 in the processing B 410 is passed from the processing B 410 to the next processing C 412.

In a case of a configuration in which the control parameter buffer 408 is secured in an external memory externally attached to an integrated circuit that is in charge of the signal processing, the control parameter is read out from the control parameter buffer 408 to an internal memory in the integrated circuit, and the control parameter in the internal memory is referred to by the synthesis processing unit 112. In addition, the control parameter in the internal memory is also referred to in the subsequent processing C 412.

In a case in which the ultrasound diagnostic apparatus does not have the synthesis processing unit 112, the processing B 410 is processing of reading out the next reception signal data from the CH memory 110 and writing the next reception signal data to the free line memory #1 or #2.

The processing C 412 represents processing of the phasing addition unit 200 and the signal processing unit 300 on the reception signal data read out from the line memory #1 or #2 of each channel 100.

In the processing C 412, the phasing addition unit 200 reads out the readout reception signal data of each channel 100 according to a plurality of different delay patterns in accordance with the control parameters and performs phasing addition to generate a reception beam signal for each pattern. This is parallel beam processing.

In addition, in the processing C 412, the signal processing unit 300 executes, for example, signal processing corresponding to the current mode on each reception beam signal output from the phasing addition unit 200 to generate an ultrasound image in the mode. The generated ultrasound image is displayed on, for example, a screen of the ultrasound diagnostic apparatus.

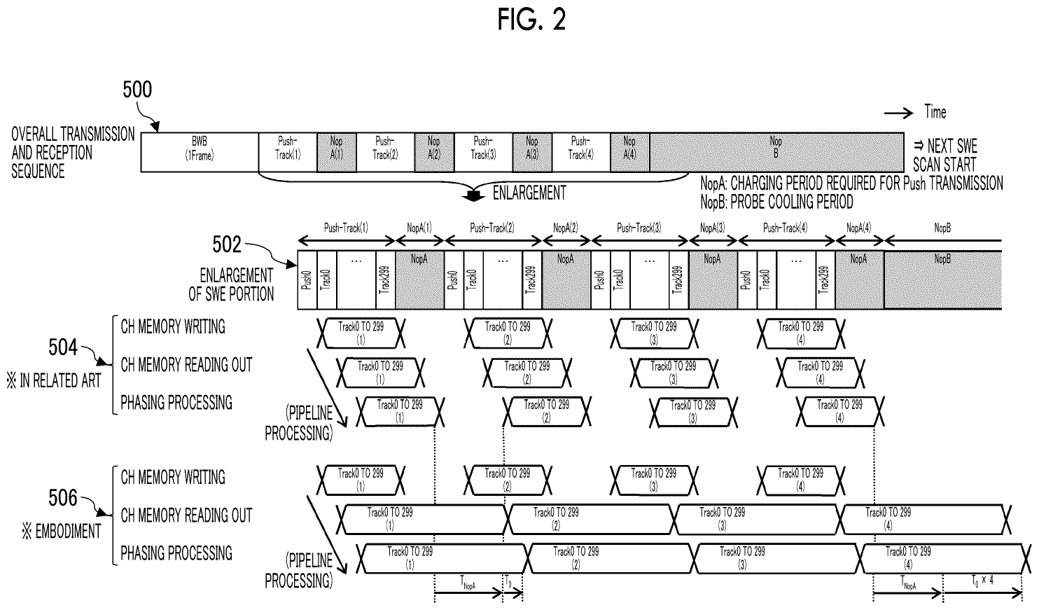

Next, an example of a temporal flow of signal processing in the SWE mode will be described with reference to FIG. 2. FIG. 2 is a diagram for describing a specific example of the reception parallel beam processing. FIG. 2 shows a time chart (also referred to as a timing chart) of the reception signal processing implemented by the ultrasound diagnostic apparatus of FIG. 1.

An uppermost chart 500 in FIG. 2 shows an overall transmission and reception sequence for one frame of image display in the SWE mode.

In the SWE mode, the SWE image is superimposed and displayed on a part of the B-mode tomographic image. Therefore, in the chart 500, first, the transmission and reception of ultrasonic waves for generating one frame of a black and white B-mode tomographic image are performed. This transmission and reception period is denoted by BWB in the drawing.

This is followed by four transmission and reception periods Push-Track (1), (2), (3), and (4) for generating the SWE image. The reason why the four transmission and reception periods are divided in this way is that a width of the SWE image that can be generated by one track pulse is small. That is, in this embodiment, a region of interest (that is, ROI) of the SWE display is divided into four small regions, and the four transmission and reception periods cover the four small regions.

An idle period NopA is provided after each of the transmission and reception periods Push-Track (1), (2), (3), and (4). The idle period NopA is a period in which the transmission and reception of the ultrasonic waves are not performed. Charging for transmitting the next push pulse is performed in the idle period NopA. The push pulse is a high-intensity ultrasonic pulse for generating a shear wave in a target tissue, and a large power needs to be supplied to the transducer 102 in order to transmit such a high-intensity pulse. The idle period NopA is a period required for charging with the large power. By way of example only, a length of one transmission and reception period Push-Track is about 100 milliseconds (ms), and a length of one idle period NopA is about 40 ms.

A chart 502 is an enlarged view of four transmission and reception periods Push-Track (1), (2), (3), and (4) for the SWE in the overall chart 500. In each transmission and reception period (for example, Push-Track (1)), first, a push pulse Push0 is transmitted, and then track pulses Track0, 1, . . . , 299 are transmitted in order. In this example, the shear wave generated in the tissue by the push pulse Push0 is tracked by 300 track pulses Track0, 1, . . . , 299. With this tracking, a propagation time (or a propagation speed) of the shear wave at each position in the tissue is obtained.

For example, in the transmission and reception period Push-Track (1) of the chart 502, a period of the push pulse Push0 corresponds to a period in which an ultrasonic wave for generating a shear wave is transmitted and received. In addition, a period of the track pulses Track0 to 299 corresponds to a period in which an ultrasonic wave for observing a shear wave is transmitted and received a plurality of times.

The idle period NopA after the fourth transmission and reception period Push-Track (4) is followed by an idle period NopB. The idle period NopB is a period for cooling the ultrasound probe heated during the four transmission and reception periods Push-Track (1) to (4), and a length thereof is, for example, about 4 seconds. In the idle period NopB, the processing for one frame is ended. After that, the same transmission and reception sequence of ultrasonic waves is performed for the next frame.

A chart group 504 shows a flow of the processing in the related art on the reception signal data obtained by the track pulse. The chart arranged in three stages shows, from top to bottom, timings of the processing of writing the data of the processing result of the second reception processing unit 108 to the CH memory 110, the processing of reading out the data from the CH memory 110, and the phasing processing of the phasing addition unit 200. As shown in the figure, the three types of processing are executed in parallel by pipeline processing.

In this example, first, after the transmission of the push pulse Push0 at the start of the transmission and reception period Push-Track (1), the track pulses Track0 to 299 are transmitted in order. Then, the reception signal for each of the track pulses Track0 to 299 is processed by the second reception processing unit 108, and the data of the processing result is written in sequence to the CH memory 110. The writing to the CH memory 110 ends immediately after the transmission and reception of the last track pulse Track299 in the transmission and reception period Push-Track (1). In the subsequent idle period NopA, the transmission and reception are not performed, and charging for the next push pulse transmission is performed. After that, the remaining three transmission and reception periods Push-Track (2), (3), and (4) and the idle period NopA are similarly repeated.

In addition, after the data of the reception signal corresponding to the first track pulse Track0 of the transmission and reception period Push-Track (1) is written to the CH memory 110, the data is read out from the CH memory 110. Thereafter, the data corresponding to the subsequent track pulses Track 1 to 299 is sequentially read out from the CH memory 110.

The sequentially readout data is temporarily held in the buffer 114 after being processed by the synthesis processing unit 112. Thereafter, the phasing addition unit 200 performs phasing processing on the data of each channel 100 to form a reception beam. The phasing processing is executed in order for the data of the track pulses Track0 to 299. The parallel beam processing is implemented by delaying a data group corresponding to one pulse in various different delay patterns and then performing the phasing processing.

In the control of the related art shown in the chart group 504, a period in which the data of the track pulses Track0 to 299 is read out from the CH memory 110 is equal to a period in which the data of 300 pulses is written to the CH memory 110. In addition, a period in which the phasing processing is performed on the data of 300 pulses read out from the CH memory 110 is also equal to the period in which the data is written to the CH memory 110.

Incidentally, in the parallel beam processing in the phasing addition unit 200, in order to form a large number of reception beams, it is necessary to perform the phasing processing many times while changing the parameters. Therefore, in order to execute the phasing processing for the same period as the data writing to the CH memory 110, a very high-speed phasing addition unit 200 is required. However, such a high-speed phasing addition unit 200 costs a lot.

On the other hand, in the present embodiment, a processing flow shown in a chart group 506 is adopted. The chart group 506 also shows the same three-stage processing flow as the above-described chart group 504 in the related art. Among these, a time chart of the writing processing on the topmost row is the same as the time chart on the topmost row of the chart group 504.

On the other hand, a period of the data readout from the CH memory 110 shown in the second stage from the top and a period of the phasing processing shown in the third stage from the top are significantly longer than the periods in the related art shown in the chart group 504. In the example of FIG. 2, a length of all of the four idle periods NopA for charging and a length of at least a part of the long idle period NopB for cooling that follows the four idle periods NopA are evenly distributed to the period of the phasing processing in the four transmission and reception periods Push-Track (1) to (4), so that the period of the phasing processing is lengthened. That is, the length of the period of the phasing processing is obtained by adding T_{NopA} and T₀ to the length of the period of the data writing to the CH memory 110 (which is approximately equal to the length of the transmission period of the track pulses Track 0 to 299). Here, T_{NopA} is a length of the idle period NopA, and T₀ is a length of ¼ of a period allocated to the extension of the period of the phasing processing in the idle period NopB. Of course, almost the entire idle period NopB may be allocated to the extension of the period of the phasing processing.

In addition, in the example of FIG. 2, as the period of the phasing processing is extended, the period of the readout processing of the data from the CH memory 110 is also extended by the same amount.

In the example of FIG. 2, since the period of the phasing processing is made considerably longer than that in the related art, a large number of reception beams can be formed by the parallel beam processing even in a configuration in which the phasing addition unit 200 that is not so fast is used.

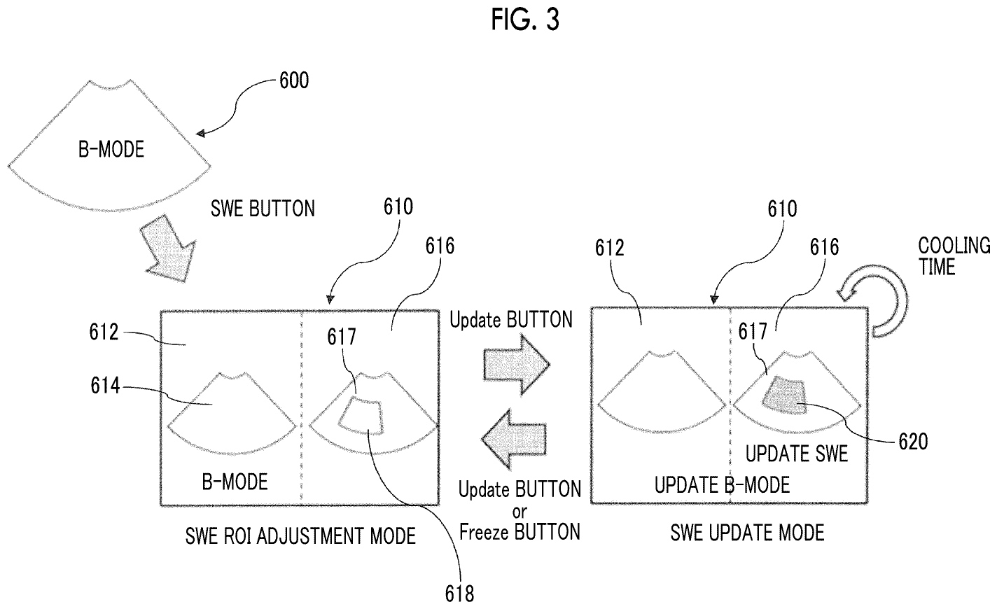

Next, an operation flow for SWE display in the ultrasound diagnostic apparatus will be exemplified with reference to FIG. 3.

As shown in FIG. 3, in a case in which a user presses an SWE button while a B-mode screen 600 is displayed on the ultrasound diagnostic apparatus, the displayed screen is switched to an SWE mode screen 610. The SWE button is a button for providing an instruction of the execution of the SWE mode, and is implemented as a mechanical button or a button on a graphical user interface.

The illustrated SWE mode screen 610 is divided into a B-mode display region 612 on a left side and an SWE display region 616 on a right side. In the B-mode display region 612, a B-mode tomographic image 614 is displayed. An SWE image 620 is displayed in the SWE display region 616. In addition, since it may be difficult to recognize which part is imaged from the SWE image 620 alone, in the illustrated example, the SWE image 620 is superimposed and displayed on a B-mode tomographic image 617. Instead of displaying the SWE image 620 in such a superimposed manner, the SWE image 620 may be displayed by replacing a corresponding portion of the B-mode tomographic image 617 with the SWE image 620.

In the example shown in the drawing, the SWE mode is divided into two sub-modes, that is, an SWE ROI adjustment mode and an SWE update mode. The SWE ROI adjustment mode is a mode in which the user sets a region of interest (ROI) to be a target of the SWE display or adjusts the set ROI. The SWE update mode is a mode in which the SWE image is displayed.

In a case in which the SWE button is pressed while the B-mode screen 600 is being displayed, the ultrasound diagnostic apparatus transitions to the SWE ROI adjustment mode. In the SWE ROI adjustment mode, the B-mode tomographic image 614 acquired in real time is displayed in the B-mode display region 612, and the same B-mode tomographic image 617 and an ROI frame 618 are displayed in the SWE display region 616. The inside of the ROI frame 618 is the ROI of the SWE display. The user adjusts a position and a size of the ROI frame 618 by operating a pointing device such as a mouse, a trackball, or a touch panel.

In a case in which a desired ROI frame 618 is obtained by the adjustment, the user can provide an instruction to perform the SWE display by pressing an Update button. In a case in which the Update button is pressed, the ultrasound diagnostic apparatus transitions to the SWE update mode. In the SWE update mode, the B-mode tomographic image 614 is displayed in the B-mode display region 612. In addition, the same B-mode tomographic image 617 is displayed in the SWE display region 616, and the SWE image 620 is superimposed or replaced and displayed in a portion of the ROI set in the SWE ROI adjustment mode in the B-mode tomographic image 617. The displayed SWE image 620 is updated at predetermined time intervals (denoted by a “cooling time” in the figure). That is, the ultrasound diagnostic apparatus executes ultrasonic operations for generating the SWE image at predetermined time intervals to generate a new SWE image, and displays the new SWE image in the SWE display region 616.

In a case in which the user presses the Update button or a Freeze button in the SWE update mode, the ultrasound diagnostic apparatus transitions to the SWE ROI adjustment mode and receives the adjustment of the ROI for the SWE from the user. The user can obtain the SWE display in the adjusted ROI by pressing the Update button again after executing the desired ROI adjustment.

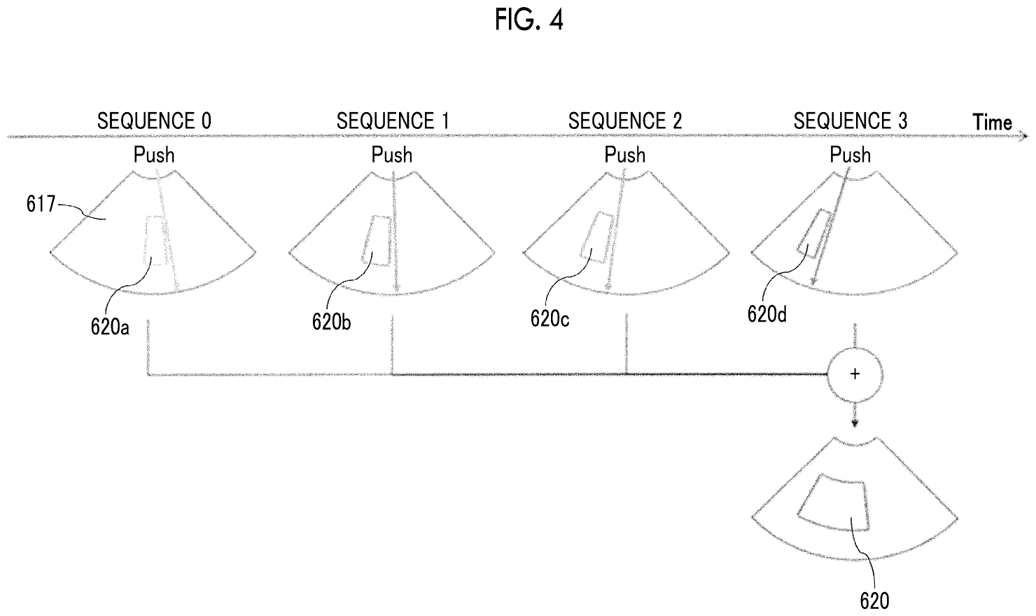

Next, a process of generating the SWE image 620 in the present embodiment will be described with reference to FIG. 4. As shown in FIG. 2, although this is merely an example, in the present embodiment, the SWE image 620 is generated by dividing one frame of the SWE image into four transmission and reception periods Push-Track.

That is, in the first transmission and reception period Push-Track (1), as shown as a sequence 0 in FIG. 4, an SWE image of a rightmost sub-region 620a of four divided regions in the ROI frame 618 in a circumferential direction is generated. That is, the push pulse Push is transmitted along a radial line of a right end of the sub-region 620a, and the SWE image is generated by about 300 track pulses after the transmission. In the subsequent transmission and reception periods Push-Track (2), (3), and (4), the SWE images of three sub-regions 620b, 620c, and 620d are generated in the order of sequences 1, 2, and 3. Then, the four sub-regions 620a to 620d are merged to generate the SWE image 620 in the ROI frame 618, and the SWE image 620 is displayed in the SWE display region 616.

As described above, in the present embodiment, the period of the phasing processing for the SWE is lengthened by using the idle period NopA or NopB, thereby improving the generation capability of the parallel beam by the phasing addition unit 200.

In addition, in the above-described embodiment, in a case in which a circuit for signal processing or control is configured as an integrated circuit, the control parameter buffer 408 can also be constructed in an external memory externally attached to the integrated circuit. In a case in which this configuration is adopted, even in a case in which an internal memory in the integrated circuit is small, a large amount of control parameters for a large number of track pulses for generating the SWE image can be held in the external memory having a larger capacity than the internal memory, and the generation of the SWE image is possible.

The embodiments described above are merely exemplary. Various modifications and improvements can be made within the scope of the disclosure described in the claims.

For example, in the above-described embodiment (see FIG. 2), the period of the phasing processing for the SWE is obtained by adding the idle period NopA to the period of writing the data to the CH memory 110 and further adding a part of the idle period NopB. However, this is just an example. Instead of this, for example, a period obtained by adding only the idle period NopA to the period of writing the data to the CH memory 110 may be used as the period of the phasing processing.

In addition, although the configuration of generating the SWE image has been described above as an example, the method of the above-described embodiment can be applied to a general configuration of generating an ultrasound image using a shear wave, in addition to the SWE.

What is claimed is:

1. An ultrasound diagnostic apparatus comprising:
a transducer array having a plurality of transducers;
a transmission and reception circuit that causes the plurality of transducers to perform transmission and reception according to a transmission and reception sequence including a first transmission and reception period in which an ultrasonic wave for generating a shear wave is transmitted and received, a second transmission and reception period in which an ultrasonic wave for observing a shear wave is transmitted and received a plurality of times, and an idle period in which no ultrasonic wave is transmitted;
a first storage unit that is provided for each transducer and stores, for each transmission of the ultrasonic waves for observing a shear wave in the second transmission and reception period, a reception signal through the transducer in response to the transmission;
a second storage unit that is provided for each transducer and has at least two line memories used to store the reception signals corresponding to different transmissions of the ultrasonic waves for observing a shear wave, the reception signals being read out from the first storage unit; and
a phasing addition unit that generates a plurality of reception beam signals by performing phasing addition for each of a plurality of different delay patterns on a set of the reception signals for the plurality of transducers corresponding to the same transmission, the set of the reception signals being read out from the line memories of the second storage unit provided for each transducer, according to the delay pattern,
wherein phasing addition unit executes a process of generating the plurality of reception beam signals corresponding to one transmission, by using at least a part of a time having a length corresponding to the idle period in addition to a time having a length corresponding to the second transmission and reception period.

2. The ultrasound diagnostic apparatus according to claim 1,
wherein the idle period includes a charging period required for transmitting the ultrasonic wave for generating a shear wave from end of the second transmission and reception period until start of the first transmission and reception period in the next transmission and reception sequence.

3. The ultrasound diagnostic apparatus according to claim 1,
wherein the idle period includes a cooling period for cooling the transducer array after the second transmission and reception period.

4. The ultrasound diagnostic apparatus according to claim 1, further comprising:
a generation unit that generates, before each of the individual transmissions in the second transmission and reception period starts, a parameter for the phasing addition for the reception signal corresponding to the transmission,
wherein the second storage unit is configured in an internal memory in the same integrated circuit as the phasing addition unit, whereas the first storage unit is configured in an external memory externally attached to the integrated circuit, and
the parameter generated by the generation unit is temporarily stored in the external memory, and is read from the external memory to the internal memory in a case of being used for the phasing addition.

5. The ultrasound diagnostic apparatus according to claim 2, further comprising:
a generation unit that generates, before each of the individual transmissions in the second transmission and reception period starts, a parameter for the phasing addition for the reception signal corresponding to the transmission,
wherein the second storage unit is configured in an internal memory in the same integrated circuit as the phasing addition unit, whereas the first storage unit is configured in an external memory externally attached to the integrated circuit, and
the parameter generated by the generation unit is temporarily stored in the external memory, and is read from the external memory to the internal memory in a case of being used for the phasing addition.

6. The ultrasound diagnostic apparatus according to claim 3, further comprising:
a generation unit that generates, before each of the individual transmissions in the second transmission and reception period starts, a parameter for the phasing addition for the reception signal corresponding to the transmission,
wherein the second storage unit is configured in an internal memory in the same integrated circuit as the phasing addition unit, whereas the first storage unit is configured in an external memory externally attached to the integrated circuit, and
the parameter generated by the generation unit is temporarily stored in the external memory, and is read from the external memory to the internal memory in a case of being used for the phasing addition.
